(19) United States
(12) Patent Application Publication
(10) Pub. No.: US 2025/0186806 A1
(43) Pub. Date: Jun. 12, 2025

(54) ULTRASONIC DIAGNOSTIC APPARATUS, ULTRASONIC DIAGNOSTIC METHOD, AND STORAGE MEDIUM

(71) Applicant: Konica Minolta, Inc., Tokyo (JP)

(72) Inventor: Kazuya TAKAGI, Tokyo (JP)

(21) Appl. No.: 18/970,301

(22) Filed: Dec. 5, 2024

(30) Foreign Application Priority Data
Dec. 7, 2023 (JP) 2023-207008

(51) Int. Cl. A61N 7/00

(52) U.S. Cl. CPC A61N 7/00; A61N 2007/0013

(57) ABSTRACT

Disclosed is an ultrasonic diagnostic apparatus including: an ultrasonic probe that transmits and receives ultrasonic waves; an ultrasonic image generator that generates an ultrasonic image by using ultrasonic waves received by the ultrasonic probe; a simulated ultrasonic image generator that generates a simulated ultrasonic image simulating ultrasonic waves emitted from a different device different from the ultrasonic diagnostic apparatus by using ultrasonic waves received by the ultrasonic probe and based on different-device information on ultrasonic irradiation of the different device; an image combiner that combines the ultrasonic image generated by the ultrasonic image generator and the simulated ultrasonic image generated by the simulated ultrasonic image generator; and a hardware processor that outputs a composite image obtained by combining by the image combiner.

### CROSS-REFERENCE TO RELATED APPLICATIONS

The entire disclosure of Japanese Patent Application No. 2023-207008, filed on Dec. 7, 2023, is incorporated herein by reference in its entirety.

### BACKGROUND OF THE INVENTION

### Technical Field

The present invention relates to an ultrasonic diagnostic apparatus, an ultrasonic diagnostic method, and a storage medium.

### Description of Related Art

There is known an ultrasonic bone fracture treatment device that promotes fracture healing by applying ultrasonic waves of weak low output pulses to a fracture part. To irradiate a fracture part with uniform ultrasonic waves while pressing a transducer against a body surface around the fracture part in an ultrasonic bone fracture treatment device. Therefore, when a fracture part is treated, it is important to specify an optimum irradiation position of ultrasonic waves on the body surface of a patient.

As a method of grasping the optimum position to be irradiated with ultrasonic waves, there is a visualization technology using an X-ray irradiation device or the like. For example, a clip or the like serving as a mark at the time of X-ray imaging is attached to a site to be irradiated by the X-ray irradiation device, and the irradiation position is determined after the positional relationship between the mark and the fracture line is confirmed. Japanese Patent No. 4944795 proposes a medical ultrasonic apparatus that grasps an optimum position by using ultrasonic waves. The medical ultrasonic apparatus compares a detection signal detected by transmitting an ultrasonic wave to a fracture part with a reference signal recorded at the time of prescription to check whether the ultrasonic wave is radiated to a prescribed position.

### SUMMARY OF THE INVENTION

However, in the technique using the conventional X-ray irradiation device or the like, there is a problem in that it is necessary to prepare a clip or the like and the apparatus is increased in size. Furthermore, there is also a problem of exposure due to reception of radiation. Furthermore, in the case of confirming whether the irradiation position of the ultrasonic waves is optimum in the conventional Japanese Patent No. 4944795, the irradiation position of the ultrasonic waves and the like are not imaged. Therefore, since a doctor or the like cannot perform work while checking an image of an irradiation position or the like of ultrasonic waves, there is a problem in that it is difficult to check an optimal irradiation position of ultrasonic waves.

Therefore, in order to solve the above-described problem, an object of the present invention is to provide an ultrasonic diagnostic apparatus, an ultrasonic diagnostic method, and a storage medium capable of efficiently performing fracture healing by simply identifying an irradiation position of an ultrasonic bone fracture treatment device.

To achieve at least one of the abovementioned objects, according to an aspect of the present invention, an ultrasonic diagnostic apparatus reflecting one aspect of the present invention is an ultrasonic diagnostic apparatus including:
an ultrasonic probe that transmits and receives ultrasonic waves;
   an ultrasonic image generator that generates an ultrasonic image by using ultrasonic waves received by the ultrasonic probe;
   a simulated ultrasonic image generator that generates a simulated ultrasonic image simulating ultrasonic waves emitted from a different device different from the ultrasonic diagnostic apparatus by using ultrasonic waves received by the ultrasonic probe and based on different-device information on ultrasonic irradiation of the different device;
   an image combiner that combines the ultrasonic image generated by the ultrasonic image generator and the simulated ultrasonic image generated by the simulated ultrasonic image generator; and
   a hardware processor that outputs a composite image obtained by combining by the image combiner.

To achieve at least one of the abovementioned objects, according to another aspect of the present invention, an ultrasonic diagnostic method reflecting one aspect of the present invention is an ultrasonic diagnostic method including:
ultrasonic image generating that is generating an ultrasonic image by using ultrasonic waves received by an ultrasonic probe;
   simulated image generating that is generating a simulated ultrasonic image simulating an irradiation signal of ultrasonic waves emitted from a different device different from an ultrasonic diagnostic apparatus by using the ultrasonic waves received by the ultrasonic probe and based on different-device information on ultrasonic irradiation of the different device;
   combining that is combining the generated ultrasonic image and the generated simulated ultrasonic image; and
   outputting that is outputting a composite image obtained by combining.

To achieve at least one of the abovementioned objects, according to another aspect of the present invention, a storage medium reflecting one aspect of the present invention is a non-transitory computer-readable storage medium storing a program causing a computer to perform:
ultrasonic image generating that is generating an ultrasonic image by using ultrasonic waves received by an ultrasonic probe;
   simulated image generating that is generating a simulated ultrasonic image simulating an irradiation signal of ultrasonic waves emitted from a different device different from an ultrasonic diagnostic apparatus by using the ultrasonic waves received by the ultrasonic probe and based on different-device information on ultrasonic irradiation of the different device;
   combining that is combining the generated ultrasonic image and the generated simulated ultrasonic image; and
   outputting that is outputting a composite image obtained by combining.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a diagram which shows an outline of a case of identifying an optimum irradiation position of ultrasonic waves for treatment which are to be emitted from an ultrasonic bone fracture treatment device, using an ultrasonic diagnostic apparatus according to a first embodiment;

FIG. 2 is a block diagram showing an example of the configuration of the ultrasonic diagnostic apparatus according to the first embodiment;

FIG. 3A is a diagram showing an example of the configuration of the ultrasonic diagnostic apparatus information according to the first embodiment;

FIG. 3B is a diagram showing an example of the configuration of the fracture treatment device information according to the first embodiment;

FIG. 4 is a flowchart illustrating an example of the operation of the ultrasonic diagnostic apparatus according to the first embodiment in a case where an irradiation position of ultrasonic waves of the ultrasonic bone fracture treatment device with respect to a fracture part of a patient is specified;

FIG. 5A is a view showing an example of an ultrasonic image generated by the image generating section according to the first embodiment;

FIG. 5B is a diagram showing an example of a simulated ultrasonic image generated by the image generating section according to the first embodiment;

FIG. 5C is a diagram illustrating an example of a composite image generated by the image processing section according to the first embodiment;

FIG. 6 is a diagram showing an example of the configuration of the ultrasonic probe of the ultrasonic diagnostic apparatus according to the first modification example of the first embodiment;

FIG. 7 is a diagram showing an example of the configuration of the ultrasonic probe of the ultrasonic diagnostic apparatus according to the second modification example of the first embodiment;

FIG. 8 is a block diagram showing an example of the configuration of the ultrasonic diagnostic apparatus according to the second embodiment;

FIG. 9 is a flowchart showing an example of the operation of the ultrasonic diagnostic apparatus in the case of specifying the irradiation position of the ultrasonic waves of the ultrasonic bone fracture treatment device with respect to the fracture part of the patient according to the second embodiment; and

FIG. 10 is a diagram illustrating an example of the configuration of the ultrasonic diagnostic apparatus according to the third embodiment.

### DETAILED DESCRIPTION

Hereinafter, one or more embodiments of the present invention will be described with reference to the drawings. However, the scope of the invention is not limited to the disclosed embodiments.

### Outline of Use Example of Ultrasonic Diagnostic Apparatus 10A and Ultrasonic Bone Fracture Treatment Device 20 According to First Embodiment

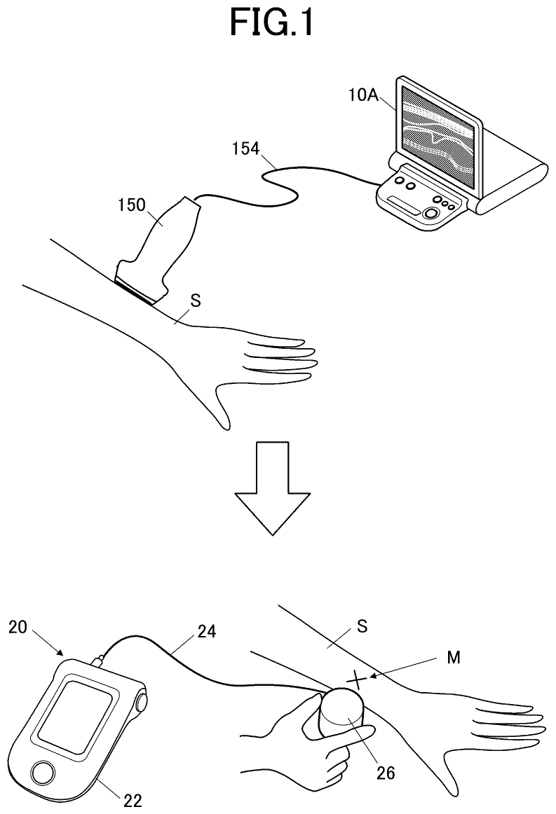

FIG. 1 is a diagram for explaining an outline of the case of determining an optimum irradiation position of therapeutic ultrasonic waves to be emitted from an ultrasonic bone fracture treatment device 20 by using an ultrasonic diagnostic apparatus 10A according to the present embodiment.

The ultrasonic bone fracture treatment device 20 is a device that promotes bone union by applying physical stimulation due to a sound pressure of ultrasonic waves to a fracture part. The ultrasonic bone fracture treatment device 20 includes a treatment device main body 22 and a transducer 26 connected via wiring 24. At home or the like, the patient applies low-output pulsed ultrasonic waves to the fracture while pressing the transducer 26 against the body surface around the fracture part. On the body surface of the subject S that is a subject to be imaged, marks M are preliminarily provided at positions to which the transducer 26 is to be pressed, that is, irradiation positions of ultrasonic waves by the transducer 26. Thus, the patient can easily specify the optimum irradiation position using the mark M as a mark, and thus can easily treat the fracture part even at home or the like.

Conventionally, an X-ray irradiation device or the like has been used to specify an optimum irradiation position by the transducer 26, but there have been problems of an increase in size of the device, exposure, and the like. Therefore, in this embodiment, the ultrasonic diagnostic apparatus 10A which is smaller than the X-ray irradiation device and has no problem of exposure to radiation is used. In an ultrasonic diagnostic apparatus 10A, a normal ultrasonic image and a simulated ultrasonic image simulating ultrasonic waves irradiated by an ultrasonic bone fracture treatment device 20 are generated at the time of examination in medical institutions such as hospitals. The ultrasonic diagnostic apparatus 10A generates a composite image by combining the generated ultrasonic image and the simulated ultrasonic image, and displays the generated composite image on the display part. Thus, a user such as a doctor can specify an optimal irradiation position of ultrasonic waves by the ultrasonic bone fracture treatment device 20 by checking the composite image. Hereinafter, an ultrasonic diagnostic apparatus 10A and the like according to the present embodiment will be described in detail.

### [Configuration Example of Ultrasonic Diagnostic Apparatus 10A]

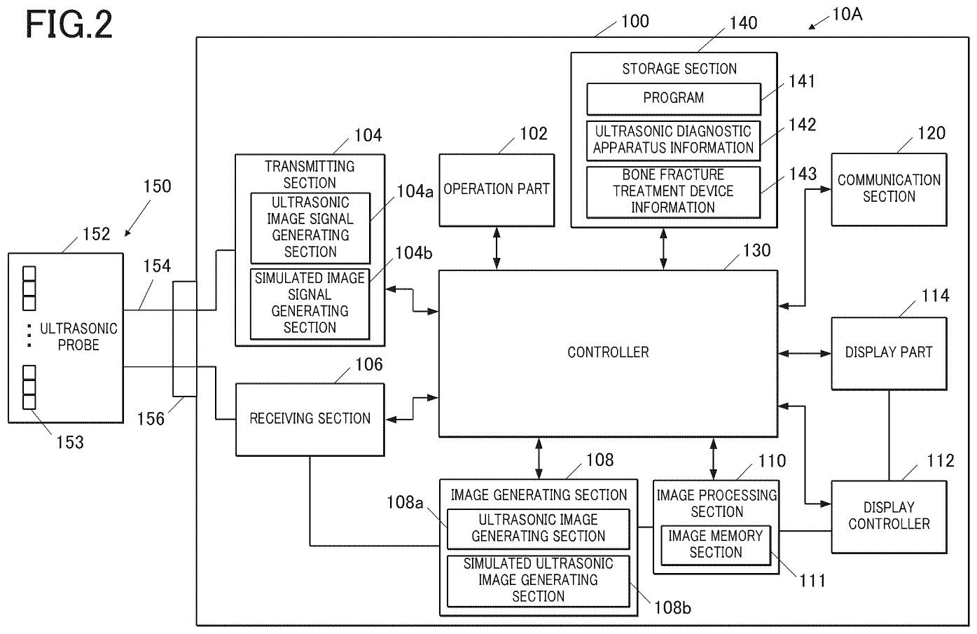

First, the ultrasonic diagnostic apparatus 10A according to the first embodiment will be described. FIG. 2 is a block diagram showing an example of the configuration of the ultrasonic diagnostic apparatus 10A according to the present embodiment. The ultrasonic diagnostic apparatus 10A is used by users such as doctors in medical facilities such as hospitals. As illustrated in FIG. 2, the ultrasonic diagnostic apparatus 10A includes an ultrasonic diagnostic apparatus main section 100 and an ultrasonic probe 150 connected to the ultrasonic diagnostic apparatus main section 100.

The ultrasonic diagnostic apparatus main section 100 includes an operation part 102, a transmitting section 104 (transmitter), a receiving section 106 (receiver), an image generating section 108, and an image processing section 110. The ultrasonic diagnostic apparatus main section 100 further includes a display controller 112, a display part 114, a communication section 120, a controller 130 (hardware processor), and a storage section 140.

The operation part 102 receives input instructions by various user operations, converts the received input instructions into electrical signals, and outputs the electrical signals to the controller 130. For example, the operation part 102 receives an input of a command for executing an installer, a parameter related to the display of an ultrasonic image, and the like. The operation part 102 includes, for example, a mouse, a keyboard, a trackball, a switch, and a button.

The operation part 102 may be, for example, a touch screen integrally combined with a display, or may be a user interface such as a microphone that receives a voice input.

The transmitting section 104 includes an ultrasonic signal generating section 104a and a simulated ultrasonic signal generating section 104b. The ultrasonic signal generating section 104a supplies a transmission (drive) signal, which is an electrical signal, to the ultrasonic probe 150 under the control of the controller 130. The ultrasonic signal generating section 104a acquires, from the storage section 140, the ultrasonic diagnostic apparatus information 142 on ultrasonic irradiation by the ultrasonic diagnostic apparatus 10A. The ultrasonic signal generating section 104a generates a transmission signal related to the ultrasonic diagnostic apparatus 10A using the frequencies, intensities, and the like of the acquired ultrasonic diagnostic apparatus information 142. The ultrasonic signal generating section 104a includes, for example, a clock generating circuit, a delay circuit, and a pulse generating circuit. The clock generation circuit generates a clock signal that determines transmission timing and a transmission frequency of a transmission signal. The delay circuit sets a delay time for each path provided in each vibrator 153 to be described later, and delays transmission of the transmission signal by the set delay time. The delay circuit focuses a transmission beam constituted by an ultrasonic wave. The pulse generating circuit generates a pulse signal as a transmission signal at a predetermined cycle. The transmitting section 104 drives, for example, a consecutive part of the plurality of transducers 153 to generate ultrasonic waves. The transmitting section 104 performs scanning while shifting the driven transducer 153 in the azimuth direction each time an ultrasonic wave is generated.

The simulated ultrasonic signal generating section 104b acquires the bone fracture treatment device information 143 on the ultrasonic irradiation of the ultrasonic bone fracture treatment device 20 from the storage section 140. The simulated ultrasonic signal generating section 104b generates a transmission signal capable of simulating ultrasonic waves to be emitted from the ultrasonic bone fracture treatment device 20, using the 10 frequencies, intensities, and the like of the acquired bone fracture treatment device information 143. The simulated ultrasonic signal generating section 104b includes a clock generation circuit, a delay circuit, a pulse generating circuit, and the like, similarly to the ultrasonic signal generating section 104a described above. Detailed description of the clock generation circuit, the delay circuit, the pulse generating circuit, and the like will be omitted.

Under the control of the controller 130, the receiving section 106 receives a reception signal, which is an electrical signal, from the ultrasonic probe 150. In the present embodiment, a reception signal corresponding to the transmission signal generated by the ultrasonic signal generating section 104a and a reception signal corresponding to the transmission signal generated by the simulated ultrasonic signal generating section 104b are received. The receiving section 106 includes, for example, an amplifier, an A/D conversion circuit, and a phasing addition circuit. The amplifier amplifies the reception signal at a preset amplification factor for each path provided in each vibrator 153. The A/D conversion circuit performs analog/digital conversion on the amplified reception signal. The phasing addition circuit gives a delay time to the A/D converted reception signal for each path provided in each transducer 153 to adjust the time phase, and adds these. The phasing addition circuit generates a sound ray signal (sound ray data) by phasing addition. Note that the receiving section 106 may include an amplifier for amplifying the reception signal.

The image generating section 108 includes an ultrasonic image generating section 108a (ultrasonic image generator) and a simulated ultrasonic image generating section 108b (simulated ultrasonic image generator). The ultrasonic image generating section 108a performs attenuation correction on the reception signals received by the receiving section 106 to correct attenuation according to transmission distances. The attenuation correction is referred to as sensitivity time control (STC). The ultrasonic image generating section 108a performs envelop detection processing, logarithmic compression, and the like on the reception signal supplied from the receiving section 106 under the control of the controller 130. The ultrasonic image generating section 108a further adjusts the dynamic range and gain of the sound ray data and converts the brightness thereof to generate B-mode image data, which is an ultrasonic image. The B-mode image data represents the intensity of a reception signal by luminance and is tomographic image information about a tissue of the subject to be inspected. The mode of the ultrasonic image is not limited to the B-mode image data. Examples of the other scan modes (image modes) include an A-mode, an M-mode, and a scan mode using the Doppler method. The Doppler method includes, for example, a color Doppler mode and a PWD. B-mode is an abbreviation for Brightness mode. A-mode is an abbreviation for Amplitude mode. M-mode is an abbreviation for Motion mode. PWD is an abbreviation for Pulsed Wave Doppler.

Unlike the ultrasonic image generating section 108a, the simulated ultrasonic image generating section 108b does not perform attenuation correction on the reception signals received by the receiving section 106. This is for accurately grasping how deep in the body the ultrasonic waves emitted from the transducer 26 of the ultrasonic bone fracture treatment device 20 reach, that is, whether the ultrasonic waves are appropriately emitted to the fracture part. The simulated ultrasonic image generating section 108b generates a simulated ultrasonic image by performing the above-described envelop detection processing, logarithmic compression, and the like, similarly to the ultrasonic image generating section 108a. The simulated ultrasonic image is, for example, B-mode image data, similarly to the ultrasonic image described above. Note that detailed description of the envelope detection processing and the like will be omitted.

Under the control of the controller 130, the image processing section 110 performs image processing on the ultrasonic image and the simulated ultrasonic image output from the image generating section 108 in accordance with various image parameters being set. The image processing section 110 includes an image memory section 111 constituted by a semiconductor memory such as a DRAM. DRAM is an abbreviation for Dynamic Random Access Memory. Under the control of the controller 130, the image processing section 110 stores the image-processed ultrasonic image and simulated ultrasonic image in the image memory section 111 on a frame-by-frame basis. In the present embodiment, the image processing section 110 generates a composite image by combining the ultrasonic image and the simulated ultrasonic image stored in the image memory section 111. The generated composite image may be stored in the image memory section 111. The image processing section 110 sequentially outputs image data such as the generated composite image to the display controller 112 according to the control of the controller 130. Note that image data in sections of frames may be referred to as ultrasonic image data or frame image data.

The display controller 112 generates an image signal for display by performing coordinate conversion or the like on the received ultrasonic image data such as a composite image under the control of the controller 130. The display controller 112 outputs the generated image signal for display to the display part 114.

Under the control of the controller 130, the display part 114 displays, on its screen, a still image or a moving image corresponding to the image signal for display output from the display controller 112. The display part 114 is, for example, a display device such as an LCD or an organic EL display. LCD is an abbreviation for Liquid Crystal Display. EL is an abbreviation for Electronic Luminescence. In the present embodiment, the display part 114 displays, on the screen, a composite image obtained by combining the ultrasonic image and the simulated ultrasonic image, based on the image signal output from the display controller 112.

The communication section 120 includes, for example, an NIC, a LAN adapter, and a communication module including a receiving section and a transmitting section. NIC is an abbreviation for Network Interface Card. The communication section 120 may be an interface to which a terminal such as a USB can be connected. The communication section 120 transmits and receives various kinds of data, information, and the like to and from, for example, an external device, an external memory, and the like. In the present embodiment, the ultrasonic diagnostic apparatus information 142, the bone fracture treatment device information 143, and the like are acquired via a memory such as a USB or an SDD, and the acquired ultrasonic diagnostic apparatus information 142 and bone fracture treatment device information 143 are output to the storage section 140. The ultrasonic diagnostic apparatus information 142 and the like may be acquired from an external device such as an information terminal connected via a network.

The controller 130 includes a processor such as a CPU, a memory such as a RAM, and the like. The CPU reads various programs 141 stored in the storage section 140, develops the programs in the RAM, and executes various processes in cooperation with the programs. The CPU may be constituted by a single processor or a plurality of processors. Specifically, the controller 130 controls the transmitting section 104, the receiving section 106, the image generating section 108, the image processing section 110, and the like by executing the program 141, thereby realizing a procedure of generating an ultrasonic image, a simulated ultrasonic image, and a composite image, a procedure of outputting a composite image, and the like.

The storage section 140 includes any storage module including, for example, an HDD, an SSD, a ROM, and a RAM. The storage section 140 stores, for example, a system program, an application program, and various types of data received by the communication section 120. The storage section 140 stores a program 141 for executing processing related to normal ultrasonic inspection, processing for identifying an optimal irradiation range of ultrasonic waves by the ultrasonic bone fracture treatment device 20, and the like. The storage section 140 stores ultrasonic diagnostic apparatus information 142 and bone fracture treatment device information 143.

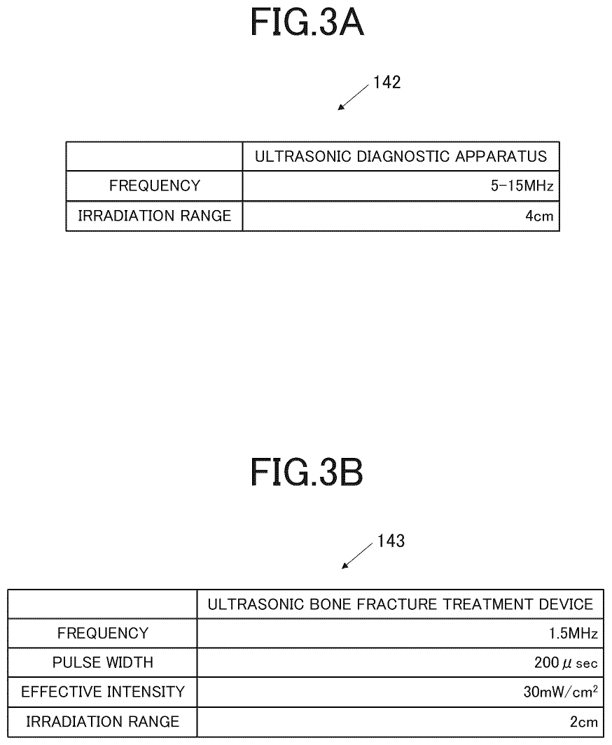

The ultrasonic diagnostic apparatus information 142 stores various conditions for generating transmission signals of ultrasonic to be emitted from the ultrasonic probe 150 of the ultrasonic diagnostic apparatus 10A. FIG. 3A shows an example of the configuration of the ultrasonic diagnostic apparatus information 142. In the ultrasonic diagnostic apparatus information 142, a transmission frequency, a pulse width, an effective intensity, and an irradiation range are stored in association with each other. For example, the transmission frequencies are 5 to 15 Mhz, and the irradiation range is 4 cm. In addition, the pulse width may be continuous or intermittent. The effective intensity may be set to a value larger than the effective intensity of the bone fracture treatment device information 143 to be described later. Note that the ultrasonic diagnostic apparatus information 142 may be frequencies, intensities, and the like obtained when actually irradiating the fracture part with ultrasonic waves using the ultrasonic diagnostic apparatus 10A in advance.

The bone fracture treatment device information 143 stores various conditions for generating a transmission signal that simulates an ultrasonic wave emitted from the transducer 26 of the ultrasonic bone fracture treatment device 20. FIG. 3B illustrates an example of the configuration of the bone fracture treatment device information 143. In the bone fracture treatment device information 143, a transmission frequency, a pulse width, an effective intensity, and an irradiation range are stored in association with each other. For example, the transmission frequency is 1.5 Mhz, the pulse width is 200 μsec, the effective intensity is 30 mW/cm², and the irradiation range is 2 cm. Since the ultrasonic used in the ultrasonic bone fracture treatment device 20 has a low output, each value of the bone fracture treatment device information 143 is smaller than each value of the ultrasonic diagnostic apparatus information 142 shown in the 3A of the drawing. Note that the bone fracture treatment device information 143 may be the frequency, intensity, and the like obtained when the ultrasonic wave is actually applied to the bone fracture part using the ultrasonic bone fracture treatment device 20 in advance.

As illustrated in FIG. 2, the ultrasonic probe 150 includes a head section 152, a cable 154, and a connector 156. The head section 152 is a portion to be pressed against the subject to be inspected. The head section 152 is provided with a plurality of vibrators 153 formed of piezoelectric elements. The transducer 153 transmits an ultrasonic wave to a subject to be inspected on the basis of a transmission signal transmitted from the ultrasonic diagnostic apparatus main section 100, and receives a reflected wave reflected off the subject to be inspected. The plurality of transducers 153 may be arranged in, for example, a one dimensional array or a two dimensional array (matrix) in the scanning direction. The number of vibrators 153 can be arbitrarily set. In the present embodiment, an electronic scanning probe of a linear scanning method is used as the ultrasonic probe 150, and ultrasonic waves are scanned by the linear scanning method. In addition to the linear scanning method, other scanning methods such as a convex scanning method and a sector scanning method may be employed.

The cable 154 has one end electrically connected to the head section 152 and the other end electrically connected to the connector 156. The connector 156 is attached to the other end of the cable 154 and is connected to the ultrasonic diagnostic apparatus main section 100. Note that the communication between the ultrasonic diagnostic apparatus main section 100 and the ultrasonic probe 150 is not limited to wired communication using the cable 154. A communication method between the ultrasonic diagnostic apparatus main section 100 and the ultrasonic probe 150 may be wireless communication using UWB or the like. UWB is an abbreviation for Ultra Wide Band.

### [Operation Example of Ultrasonic Diagnostic Apparatus 10A]

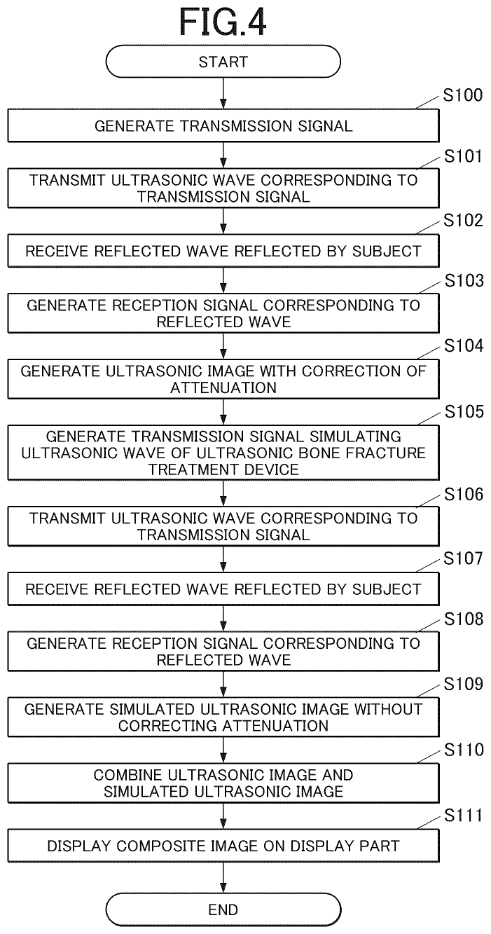

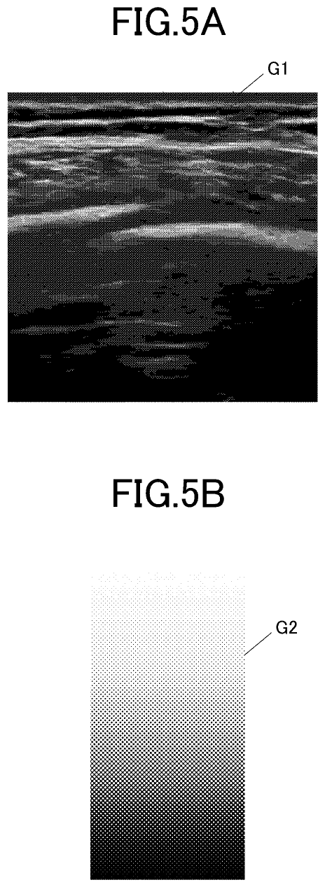

Next, the flow of the ultrasonic diagnostic method in the case of specifying the irradiation position of the ultrasonic bone fracture treatment device 20 with respect to the fracture part of the patient according to the first embodiment will be described. FIG. 4 is a flowchart showing an example of the operation of the ultrasonic diagnostic apparatus 10A in the case of specifying the irradiation position of the ultrasonic waves of the ultrasonic bone fracture treatment device 20 with respect to the fracture part of the patient according to the first embodiment. The diagram 5A is a diagram illustrating an example of the ultrasonic image G1 generated by the image generating section 108. FIG. 5B is a diagram illustrating an example of the simulated ultrasonic image G2 generated by the image generating section 108. FIG. 5C is a diagram illustrating an example of the composite image G3 generated by the image processing section 110. In FIG. 5C, the simulated ultrasonic image G2 is indicated by a two dot chain line.

The transmitting section 104 generates a transmission signal of ultrasonic waves to be emitted from the ultrasonic probe 150 of the ultrasonic diagnostic apparatus 10A (step S100). Specifically, the transmitting section 104 generates a transmission signal on the basis of the transmission frequency, the pulse width, the effective intensity, and the like included in the ultrasonic diagnostic apparatus information 142 acquired from the storage section 140.

The ultrasonic probe 150 irradiates the subject S with ultrasonic waves based on the transmission signals generated by the transmitting section 104 (step S101). The ultrasonic probe 150 receives the reflected wave reflected off a tissue or the like in the body of the subject S (step S102).

The receiving section 106 generates a reception signal based on the reflected wave received by the ultrasonic probe 150 (step S103). Specifically, the receiving section 106 generates reception signals by performing processing such as analog/digital conversion and phasing addition on the reflected waves received by the ultrasonic probe 150.

The image generating section 108 generates an ultrasonic image related to the ultrasonic diagnostic apparatus 10A by performing attenuation correction processing or the like on the reception signal generated by the receiving section 106 (step S104). To be more specific, as shown in FIG. 5A, since the attenuation of the reception signal is corrected and the gain in the deep portion is increased in the ultrasonic image G1, the brightness is uniformly adjusted as a whole. The width-direction size of the ultrasonic image G1 is a width W1 corresponding to an irradiation range by the ultrasonic probe 150. The ultrasonic image G1 includes, for example, a fracture part (not illustrated) of the subject S. Note that step S104 corresponds to an ultrasonic image generating step.

The transmitting section 104 generates a transmission signal that simulates ultrasonic waves emitted from the transducer of the ultrasonic bone fracture treatment device (S105). Specifically, the transmitting section 104 generates a transmission signal on the basis of the transmission frequency, the pulse width, the effective intensity, and the like included in the bone fracture treatment device information 143 acquired from the storage section 140.

The ultrasonic probe 150 irradiates the subject S with ultrasonic waves based on the transmission signals generated by the transmitting section 104 (step S106). The ultrasonic probe 150 receives the reflected wave reflected off the inside of the body of the subject S or the like (step S107).

The receiving section 106 generates a reception signal based on the reflected wave received by the ultrasonic probe 150 (step S108). Specifically, the receiving section 106 performs processing such as analog/digital conversion and phasing addition on the reflected wave received by the ultrasonic probe 150 to generate a reception signal related to the ultrasonic bone fracture treatment device 20.

The image generating section 108 generates a simulated ultrasonic image without correcting the attenuation of the reception signals generated by the receiving section 106 (step S109). To be more specific, as shown in FIG. 5B, since the attenuation of the reception signal is not corrected in the simulated ultrasonic image G2, the brightness, which is the gain, decreases according to the propagation length, that is, as the depth increases. The width-direction size of the simulated ultrasonic image G2 is a width W2 corresponding to an irradiation range by the transducer 26 of the ultrasonic bone fracture treatment device 20, and is narrower than the width W1 of the above-described ultrasonic image G1. Note that step S109 corresponds to a simulated image generating step.

The image processing section 110 combines the ultrasonic image data corresponding to the ultrasonic image G1 generated by the image generating section 108 and the simulated ultrasonic image data corresponding to the simulated ultrasonic image G2 (step S110). To be specific, the image processing section 110 acquires the irradiation range of the ultrasonic image G1 from the ultrasonic diagnostic apparatus information 142, and acquires the irradiation range of the simulated ultrasonic image G2 from the bone fracture treatment device information 143. The image processing section 110 aligns the positions of the ultrasonic image G1 and the simulated ultrasonic image G2 based on the acquired irradiation range of the ultrasonic image G1 and the irradiation range of the simulated ultrasonic image G2. In this case, as illustrated in the 5C of the drawing, the image processing section 110 aligns the centers of the widths W2 of the simulated ultrasonic images G2 with the centers of the widths W1 of the ultrasonic images G1. In this way, the image processing section 110 generates a composite image G3 by combining the ultrasonic image G1 and the simulated ultrasonic image G2. Note that Step S110 corresponds to a combining step.

The controller 130 functions as an outputter, and outputs the composite image G3 generated by the image processing section 110 to the display part 114, the display controller 112, and the like (step S111). The step S111 corresponds to an outputting step. The display part 114 displays the output composite image G3 on a screen. Thus, a user such as a doctor can confirm whether an irradiation range of ultrasonic waves by the ultrasonic bone fracture treatment device 20 corresponds to a fracture part by viewing the composite image G3. Furthermore, the user can confirm whether the ultrasonic wave from the ultrasonic bone fracture treatment device 20 reaches the depth of the fracture part by viewing the composite image G3. Note that in the case of generating a plurality of frames, a series of processing as described above is repeatedly executed.

According to the first embodiment, since the ultrasonic diagnostic apparatus 10A generates the reception signal and the transmission signal simulating the ultrasonic waves emitted from the ultrasonic bone fracture treatment device 20, it is possible to realize the imaging of the ultrasonic waves by the ultrasonic bone fracture treatment device 20 by using these signals. Furthermore, according to the first embodiment, the composite image is generated by combining the ultrasonic image related to the ultrasonic diagnostic apparatus 10A and the simulated ultrasonic image related to the ultrasonic bone fracture treatment device 20. Thus, it is possible to simulatively confirm whether the ultrasonic waves emitted from the ultrasonic bone fracture treatment device 20 are emitted to the fracture part, and therefore, it is possible to specify an optimum irradiation position of the fracture part or the like with the ultrasonic waves emitted from the ultrasonic bone fracture treatment device 20.

Furthermore, according to the present embodiment, the optimal irradiation position of the ultrasonic bone fracture treatment device 20 can be specified by the ultrasonic diagnostic apparatus 10A without using an X-ray irradiation device or the like. Thus, it is possible to avoid the problem of radiation exposure, and it is not necessary to prepare a clip or the like to be used when the irradiation position is specified. In addition, since the optimal irradiation position of the ultrasonic bone fracture treatment device 20 can be specified without using a large X-ray irradiation device or the like, it is possible to improve the efficiency of the work.

### First Modification Example of First Embodiment

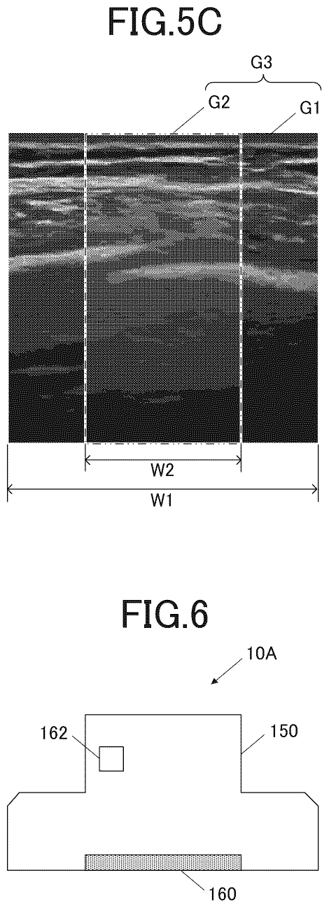

Next, a first modification example according to the first embodiment will be described. FIG. 6 shows an example of the configuration of the ultrasonic probe 150 of the ultrasonic diagnostic apparatus 10A according to the first modification example of the first embodiment.

As shown in FIG. 6, visual information 160 indicating an irradiation range of ultrasonic waves by the transducer 26 of the ultrasonic bone fracture treatment device 20 is attached to the surface on the distal end side of the ultrasonic probe 150. The visual information 160 is a mark for directly recognizing an irradiation range of ultrasonic waves by the transducer 26. The visual information 160 can be used as information for complementing a means for specifying the irradiation range of the ultrasonic of the ultrasonic bone fracture treatment device 20 by visually recognizing the composite image G3 displayed on the display part 114. For example, the visual information 160 is formed with a rectangular mark indicating the size in the width direction of the irradiation range of the ultrasonic waves by the transducer 26, and is attached to a side edge portion on the tip end side of the ultrasonic probe 150. The visual information 160 may be formed by, for example, a method of coloring with a marker or the like, a method of sticking a seal, or other known methods. Thus, in a state where the ultrasonic probe 150 is pressed against the body surface, the ultrasonic irradiation range of the transducer 26 can be intuitively recognized by visually recognizing the visual information 160.

As shown in FIG. 6, an angle sensor 162 may be mounted on the ultrasonic probe 150. The angle sensor 162 functions as a posture acquisition section (posture acquirer). The angle sensor 162 detects an angle of the ultrasonic probe 150 when the ultrasonic probe 150 is pressed against the body surface located at the fracture part of the subject S. The ultrasonic diagnostic apparatus 10A may display the detection result of the angle of the ultrasonic probe 150 by the angle sensor 162 on the screen of the display part 114. Thus, the user can recognize an irradiation range of ultrasonic waves by the transducer 26 while confirming the inclination of the ultrasonic probe 150 in a state where the ultrasonic probe 150 is pressed against the body surface. Furthermore, the degree of pressing of the ultrasonic probe 150 against the body surface or the like can be grasped from the angle of the ultrasonic probe 150.

### Second Modification Example of First Embodiment

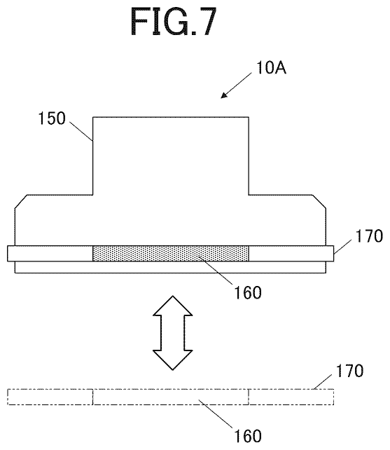

The above-described visual information 160 may be of an attachment type that is detachable from the ultrasonic probe 150. FIG. 7 shows an example of the configuration of the ultrasonic probe 150 and the attachment 170 of the ultrasonic diagnostic apparatus 10A according to the second modification example of the first embodiment.

As illustrated in FIG. 7, the attachment 170 is removably attached to the tip part of the ultrasonic probe 150. The attachment 170 is formed of, for example, a frame body, and is attached to a position that does not affect transmission and reception of ultrasonic waves, for example, an outer peripheral portion of the vibrator 153. The above-described visual information 160 is provided on the attachment 170. The center of the visual information 160 in the width direction is located at the center of the attachment 170 in the width direction.

With such a configuration, when a normal inspection is performed, the ultrasonic probe 150 can be used in a state where the attachment 170 is removed. On the other hand, when the irradiation range of the ultrasonic waves by the ultrasonic bone fracture treatment device 20 is confirmed, the attachment 170 is attached to the ultrasonic probe 150, and thus it is possible to grasp the irradiation range by the transducer 26 of the ultrasonic bone fracture treatment device 20 in a bath-dry manner.

### Second Embodiment

The second embodiment is different from the first embodiment in that the ultrasonic emitted from the transducer 26 of the ultrasonic bone fracture treatment device 20 is simulated from the reception signal of the reflected wave of the ultrasonic diagnostic apparatus 10B. In the following description, differences from the first embodiment will be mainly described, and description of points common to the first embodiment will be omitted. In the description of the second embodiment, parts common to the first embodiment are denoted by the same reference numerals.

### [Configuration Example of Ultrasonic Diagnostic Apparatus 10B]

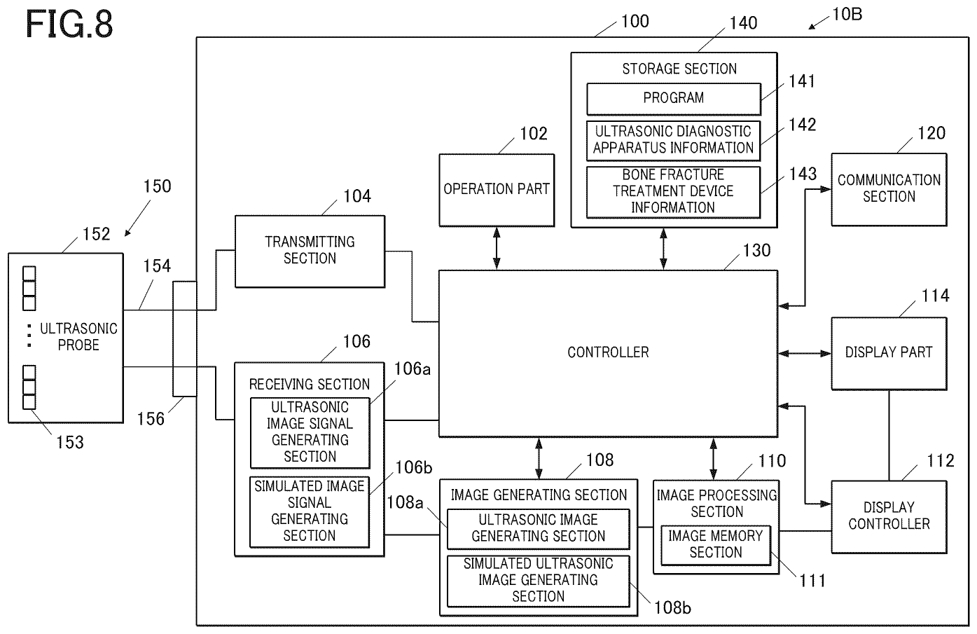

First, the ultrasonic diagnostic apparatus 10B according to the second embodiment will be described. FIG. 8 is a block diagram showing an example of the configuration of the ultrasonic diagnostic apparatus 10B according to the second embodiment.

As shown in FIG. 8, the ultrasonic diagnostic apparatus 10B includes an ultrasonic diagnostic apparatus main section 100 and an ultrasonic probe 150 connected to the ultrasonic diagnostic apparatus main section 100. The ultrasonic diagnostic apparatus main section 100 includes an operation part 102, a transmitting section 104, a receiving section 106, an image generating section 108, and an image processing section 110. The ultrasonic diagnostic apparatus main section 100 further includes a display controller 112, a display part 114, a communication section 120, a controller 130, and a storage section 140.

In the second embodiment, the functions of the ultrasonic signal generating section 104a and the simulated ultrasonic signal generating section 104b of the transmitting section 104 of the first embodiment are executed on the receiving section 106 side. The receiving section 106 includes an ultrasonic signal generating section 106a and a simulated ultrasonic signal generating section 106b. The ultrasonic signal generating section 106a performs processing such as amplification, analog/digital conversion, and phasing addition on the reception signal received from the ultrasonic probe 150.

The simulated ultrasonic signal generating section 106b acquires the bone fracture treatment device information 143 on the ultrasonic bone fracture treatment device 20 from the storage section 140. The simulated ultrasonic signal generating section 106b generates a reception signal simulating a reflected wave reflected by the subject S when ultrasonic waves are emitted from the ultrasonic bone fracture treatment device 20, using the frequencies, the effective intensities, the irradiation ranges, and the like of the acquired bone fracture treatment device information 143. In addition, the simulated ultrasonic signal generating section 106b performs processing such as amplification, analog/digital conversion, and phasing addition on the reception signal.

The image generating section 108 includes an ultrasonic image generating section 108a and a simulated ultrasonic image generating section 108b. The ultrasonic image generating section 108a performs attenuation correction on the reception signals received by the receiving section 106 to correct attenuation according to transmission distances. The ultrasonic image generating section 108a performs envelop detection processing, logarithmic compression, and adjustment of dynamic range and gain on the sound ray signals supplied from the receiving section 106 to perform brightness conversion, and generates ultrasonic image data.

The simulated ultrasonic image generating section 108b generates a simulated ultrasonic image by performing the above-described envelop detection processing, logarithmic compression, and the like on the reception signals received by the receiving section 106 without performing attenuation correction thereon, unlike the ultrasonic image generating section 108a. Note that detailed description of the envelope detection processing and the like will be omitted. The image processing section 110 generates a composite image by combining the generated ultrasonic image and the simulated ultrasonic image. The display part 114 displays the generated composite image on a screen.

### [Operation Example of Ultrasonic Diagnostic Apparatus 10B]

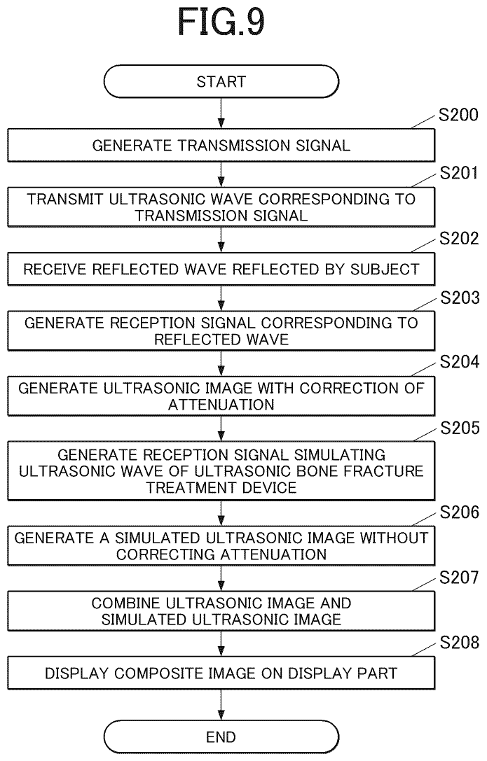

FIG. 9 is a flowchart showing an example of the operation of the ultrasonic diagnostic apparatus 10B in the case of specifying the irradiation position of the ultrasonic waves of the ultrasonic bone fracture treatment device with respect to the fracture part of the patient according to the second embodiment. Note that a detailed description of common contents with the steps described in FIG. 4 of the first embodiment will be omitted.

The transmitting section 104 generates a transmission signal of ultrasonic waves to be emitted from the ultrasonic probe 150 of the ultrasonic diagnostic apparatus 10B (step S200). Specifically, the transmitting section 104 generates a transmission signal on the basis of the transmission frequency, the pulse width, the effective intensity, and the like included in the ultrasonic diagnostic apparatus information 142 acquired from the storage section 140.

The ultrasonic probe 150 irradiates the subject S with ultrasonic waves based on the transmission signals generated by the transmitting section 104 (step S201). The ultrasonic probe 150 receives the reflected wave reflected off a tissue or the like in the body of the subject S (step S202). The receiving section 106 generates a reception signal based on the reflected wave received by the ultrasonic probe 150 (step S203).

The image generating section 108 generates an ultrasonic image related to the ultrasonic diagnostic apparatus 10B by performing attenuation correction processing or the like on the reception signal generated by the receiving section 106 (step S204). To be specific, as illustrated in 5A of the figure, the image generating section 108 generates the ultrasonic image G1 which has a width W1 corresponding to the irradiation range by the ultrasonic probe 150 and in which the brightness is adjusted to be uniform as a whole by the attenuation correction.

The receiving section 106 uses the reflected waves received by the ultrasonic probe 150 to generate a reception signal that simulates the reflected waves reflected by the subject S when ultrasonic is emitted from the transducer 26 of the ultrasonic bone fracture treatment device 20 (step S205). That is, the receiving section 106 generates a reception signal for the ultrasonic bone fracture treatment device 20 by performing predetermined processing on the same reflected wave as in the case of generating a normal ultrasonic image. For example, the receiving section 106 generates a reception signal related to the ultrasonic bone fracture treatment device 20 on the basis of the effective intensity, the irradiation range, and the like included in the bone fracture treatment device information 143 acquired from the storage section 140.

The image generating section 108 generates a simulated ultrasonic image without correcting the attenuation of the reception signals generated by the receiving section 106 (step S206). To be specific, as illustrated in 5B of the figure, the image generating section 108 generates the simulated ultrasonic image G1 that is narrower than the ultrasonic image G2 and has brightness lowered according to the propagation distances of the ultrasonic waves.

The image processing section 110 combines the ultrasonic image data corresponding to the ultrasonic image G1 generated by the image generating section 108 and the simulated ultrasonic image data corresponding to the simulated ultrasonic image G2 (step S207). To be specific, the image processing section 110 aligns the positions of the ultrasonic image G1 and the simulated ultrasonic image G2 by using the irradiation range of the ultrasonic image G1 and the irradiation range of the simulated ultrasonic image G2.

The controller 130 outputs the composite image G3 generated by the image processing section 110 to the display part 114, the display controller 112, and the like (step S208). The display part 114 displays the output composite image G3 on a screen. Thus, a user such as a doctor can confirm whether an irradiation range of ultrasonic waves by the ultrasonic bone fracture treatment device 20 corresponds to a fracture part by viewing the composite image G3. Furthermore, the user can confirm whether the ultrasonic waves from the ultrasonic bone fracture treatment device 20 have reached the depth of the fracture part by viewing the composite image G3. Note that in the case of generating a plurality of frames, a series of processing as described above is repeatedly executed.

According to the second embodiment, the same operation and effect as those of the first embodiment described above can be achieved. Specifically, by generating a composite image of the ultrasonic image and the simulated ultrasonic image, it is possible to specify an optimal irradiation position of the fracture part or the like with the ultrasonic waves emitted from the ultrasonic bone fracture treatment device 20. Furthermore, in the second embodiment, unlike the first embodiment, a transmission signal for the ultrasonic bone fracture treatment device 20 is not generated on the transmission side, but a reception signal for the ultrasonic bone fracture treatment device 20 is generated on the reception side. That is, the ultrasonic image and the simulated ultrasonic image are generated using the common reflected wave received by the receiving section 106. Thus, since it is not necessary to generate a transmission signal for the ultrasonic bone fracture treatment device 20 at the time of transmission of ultrasonic waves, an ultrasonic image or the like can be generated without reducing the frame rate.

### Third Embodiment

In the third embodiment, the transducer 26 of the ultrasonic bone fracture treatment device 20 is attached to the ultrasonic probe 150, thereby identifying an irradiation range of ultrasonic waves by the ultrasonic bone fracture treatment device 20. In the following description, differences from the first embodiment will be mainly described, and description of points common to the first embodiment will be omitted. In addition, in the description of the third embodiment, portions common to the first embodiment will be described with the same reference numerals.

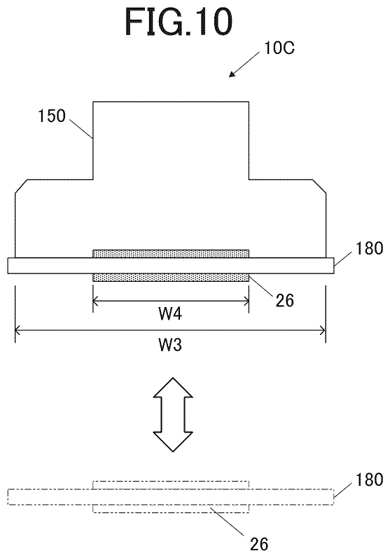

FIG. 10 shows an example of the configuration of the ultrasonic diagnostic apparatus 10C according to the third embodiment. The transducer 26 of the ultrasonic bone fracture treatment device 20 is removably attached to the attachment 180. The attachment 180 is detachably attached to the distal end side of the ultrasonic probe 150 of the ultrasonic diagnostic apparatus 10C. A width W4 of the transducer 26 is smaller than a width W3 of the ultrasonic probe 150. The transducer 26 is attached to the ultrasonic probe 150 such that the center of the transducer 26 in the width direction coincides with the center of the ultrasonic probe 150 in the width direction. The ultrasonic probe 150 or the attachment 180 may be provided with an angle sensor for detecting the angle of the ultrasonic probe 150.

In a case where an irradiation range of ultrasonic waves by the ultrasonic bone fracture treatment device 20 is specified, both of the ultrasonic diagnostic apparatus 10C and the ultrasonic bone fracture treatment device 20 are driven in a state where the attachment 180 is attached to the tip part of the ultrasonic probe 150. Thus, ultrasonic waves are emitted from each of the ultrasonic probe 150 and the transducer 26. The ultrasonic probe 150 receives a reflected wave reflected inside the body of the subject S. The reflected waves include interference waves generated by interference between the ultrasonic waves emitted from the transducer 26 and the ultrasonic waves emitted from the ultrasonic probe 150. The ultrasonic diagnostic apparatus 10C generates an ultrasonic image based on reception signals of the received reflected waves. An ultrasonic wave (interference wave) of the columnar transducer 26 appears in the ultrasonic image. For example, the generated ultrasonic image is an image in which the simulated ultrasonic image G1 in the middle of the ultrasonic images G2 illustrated in the 5C of the drawing is replaced with an image in which interfering waves are rendered. The ultrasonic diagnostic apparatus 10C displays the generated ultrasonic image on the screen of the display part 114.

According to the third embodiment, the user such as the doctor can confirm whether the irradiation range of the ultrasonic waves by the ultrasonic bone fracture treatment device 20 coincides with the fracture part by viewing the ultrasonic image on the display part 114. Furthermore, the user can confirm whether the ultrasonic waves from the ultrasonic bone fracture treatment device 20 have reached the depth of the fracture part by viewing the composite image G3.

Since the simulated ultrasonic image which simulates the ultrasonic waves emitted from the different device is combined with the ultrasonic image in which the fracture part or the like is reflected According to an aspect of the embodiments, it is possible to specify the optimum irradiation position of the fracture part or the like with the ultrasonic waves emitted from the different device.

Although the preferred embodiments of the present disclosure have been described in detail with reference to the accompanying drawings, the technical scope of the present disclosure is not limited to such examples. Furthermore, those to which various modification examples and improvements have been applied naturally belong to the technical scope of the present disclosure within the category of the technical idea described in the scope of the claims of those skilled in the art.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2023-207008, filed on Dec. 7, 2023, including description, claims, drawings and abstract is incorporated herein by reference.

What is claimed is:

1. An ultrasonic diagnostic apparatus comprising:
an ultrasonic probe that transmits and receives ultrasonic waves;
an ultrasonic image generator that generates an ultrasonic image by using ultrasonic waves received by the ultrasonic probe;
a simulated ultrasonic image generator that generates a simulated ultrasonic image simulating ultrasonic waves emitted from a different device different from the ultrasonic diagnostic apparatus by using ultrasonic waves received by the ultrasonic probe and based on different-device information on ultrasonic irradiation of the different device;
an image combiner that combines the ultrasonic image generated by the ultrasonic image generator and the simulated ultrasonic image generated by the simulated ultrasonic image generator; and
a hardware processor that outputs a composite image obtained by combining by the image combiner.

2. The ultrasonic diagnostic apparatus according to claim 1, wherein the different device is an ultrasonic bone fracture treatment device.

3. The ultrasonic diagnostic apparatus according to claim 1, wherein the different-device information includes at least one or more of a waveform, an intensity, and an irradiation range of ultrasonic waves to be emitted from the different device.

4. The ultrasonic diagnostic apparatus according to claim 1, further comprising a transmitter that generates a transmission signal simulating an ultrasonic wave of the different device based on the different-device information.

5. The ultrasonic diagnostic apparatus according to claim 1, further comprising a receiver that generates, from an ultrasonic wave received by the ultrasonic probe, a reception signal simulating an ultrasonic wave of the different device based on the different-device information.

6. The ultrasonic diagnostic apparatus according to claim 1, wherein the image combiner combines the ultrasonic image and the simulated ultrasonic image based on irradiation range information of the ultrasonic waves in the different-device information.

7. The ultrasonic diagnostic apparatus according to claim 1, further comprising a display part that displays the composite image output by the hardware processor on a screen.

8. The ultrasonic diagnostic apparatus according to claim 1, wherein the ultrasonic probe is provided with visual information visually indicating a size of an ultrasonic irradiator of the different device.

9. The ultrasonic diagnostic apparatus according to claim 1, wherein the ultrasonic probe is provided with a posture acquirer that acquires information on a posture of the ultrasonic probe.

10. The ultrasonic diagnostic apparatus according to claim 8, wherein the visual information is detachably attached to the ultrasonic probe.

11. An ultrasonic diagnostic method comprising:
ultrasonic image generating that is generating an ultrasonic image by using ultrasonic waves received by an ultrasonic probe;
simulated image generating that is generating a simulated ultrasonic image simulating an irradiation signal of ultrasonic waves emitted from a different device different from an ultrasonic diagnostic apparatus by using the ultrasonic waves received by the ultrasonic probe and based on different-device information on ultrasonic irradiation of the different device;
combining that is combining the generated ultrasonic image and the generated simulated ultrasonic image; and
outputting that is outputting a composite image obtained by combining.

12. A non-transitory computer-readable storage medium storing a program causing a computer to perform:
ultrasonic image generating that is generating an ultrasonic image by using ultrasonic waves received by an ultrasonic probe;
simulated image generating that is generating a simulated ultrasonic image simulating an irradiation signal of ultrasonic waves emitted from a different device different from an ultrasonic diagnostic apparatus by using the ultrasonic waves received by the ultrasonic probe and based on different-device information on ultrasonic irradiation of the different device;
combining that is combining the generated ultrasonic image and the generated simulated ultrasonic image; and
outputting that is outputting a composite image obtained by combining.
